(19) United States
(12) Patent Application Publication
(10) Pub. No.: US 2010/0256455 A1
(43) Pub. Date: Oct. 7, 2010

(54) Hand-held illuminating device for finding blood vessel

(76) Inventors: Tsu-I Hsia, Taipei (TW); Hsiu-Chen Yu, Taipei (TW); Chin-Liang Lin, Taipei (TW)

Correspondence Address: SAM CHEN, 7F-1, 293, ROOSEVELT ROAD, SEC 3, TAIPEI, TW

(21) Appl. No.: 12/625,564

(22) Filed: Nov. 25, 2009

(30) Foreign Application Priority Data
Apr. 6, 2009 (TW) 098205499

(51) Int. Cl. A61B 1/06

(52) U.S. Cl. 600/249

(57) ABSTRACT

A hand-held illuminating device for finding a blood vessel (e.g., vein) for blood transfusion includes a main body adapted to be stably held by the hand; and a battery powered illumination assembly in the main body and comprising a circuit board, a plurality of LEDs mounted on the circuit board and electrically connected thereto, a dim switch on an outer surface of the main body for activating the LEDs or not, and a lens on one end of the main body in proximity to the LEDs. The LEDs comprises a plurality of red LEDs capable of emitting red light having a wavelength in the range of 590 mm to 660 mm, and a plurality of orange LEDs capable of emitting orange light having a wavelength in the range of 590 mm to 660 mm.

### BACKGROUND OF THE INVENTION

1. Field of Invention

The invention relates to illuminating devices and more particularly to a hand-held illuminating device for finding a blood vessel (e.g., vein) so as to facilitate, for example, blood transfusion.

2. Description of Related Art

Conventionally, a medical employee may insert a catheter into a vein in blood transfusion. However, finding the vein is not easy without any auxiliary illumination means, especially for fat people.

Thus, it is desirable to provide a novel hand-held illuminating device for finding a blood vessel (e.g., vein) so as to facilitate, for example, blood transfusion.

### SUMMARY OF THE INVENTION

It is therefore one object of the invention to provide a hand-held illuminating device for finding a blood vessel for facilitating blood transfusion.

The above and other objects, features and advantages of the invention will become apparent from the following detailed description taken with the accompanying drawings.

### BRIEF DESCRIPTION OF THE DRAWINGS

FIG. 1 is a perspective view of a first preferred embodiment of hand-held illuminating device for finding blood vessel for blood transfusion according to the invention;

FIG. 2 is a longitudinal sectional view of the illuminating device of FIG. 1 with the lens removed;

FIG. 3 is a top plan view of FIG. 1;

FIG. 4 is a perspective view of a second preferred embodiment of hand-held illuminating device for finding blood vessel for blood transfusion according to the invention;

FIG. 5 is an environmental view of the illuminating device being held by a medical employee;

FIG. 6 is a side elevation of a third preferred embodiment of hand-held illuminating device for finding blood vessel for blood transfusion according to the invention;

FIG. 7 is a front view of the illuminating device of FIG. 6;

FIG. 8 is a top plan view of the illuminating device of FIG. 6 with the lens removed;

FIG. 9 is a side elevation of a fourth preferred embodiment of hand-held illuminating device for finding blood vessel for blood transfusion according to the invention;

FIG. 10 is a front view of the illuminating device of FIG. 9;

FIG. 11 is a top plan view of the illuminating device of FIG. 9 with the lens removed;

FIG. 12 is a side elevation of a fifth preferred embodiment of hand-held illuminating device for finding blood vessel for blood transfusion according to the invention; and

FIG. 13 is a top plan view of the illuminating device of FIG. 12 with the lens removed.

### DETAILED DESCRIPTION OF THE INVENTION

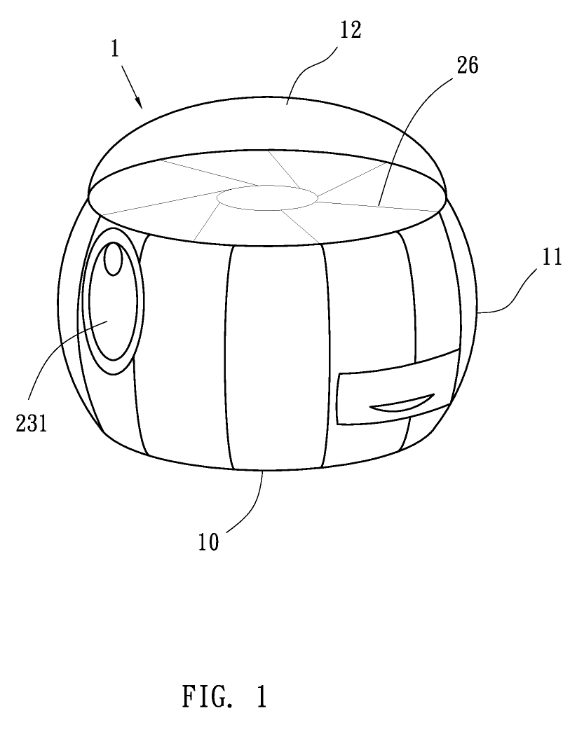

Referring to FIGS. 1 to 3, a hand-held illuminating device for finding blood vessel (e.g., vein) for blood transfusion thereafter in accordance with a first preferred embodiment of the invention comprises the following components as discussed in detail below.

A main body 10 is shaped to be stably held by the hand. The main body 10 has an outer surface 11 with a plurality of parallel convex sections other than top and bottom. The provision of the convex sections on the outer surface 11 can increase traction with the fingers and the main body 10.

An illumination assembly 20 is assembled in the main body 10 and comprises a plurality of cells 21 proximate bottom, an upper circuit board 22 electrically connected to the cells 21, a plurality of LEDs (light-emitting diodes) 23 mounted on a top surface of the circuit board 22 and electrically connected thereto, a dim switch 231 on the outer surface of the main body 10 and electrically connected to the cells 21 so that the LEDs 23 can be activated or deactivated by manipulating the dim switch 231, a control circuitry (not shown) formed in the circuit board 22, a top aperture 24 above the LEDs 23, and a control 241 on the outer surface of the main body 10 for adjusting diameter of the aperture 24.

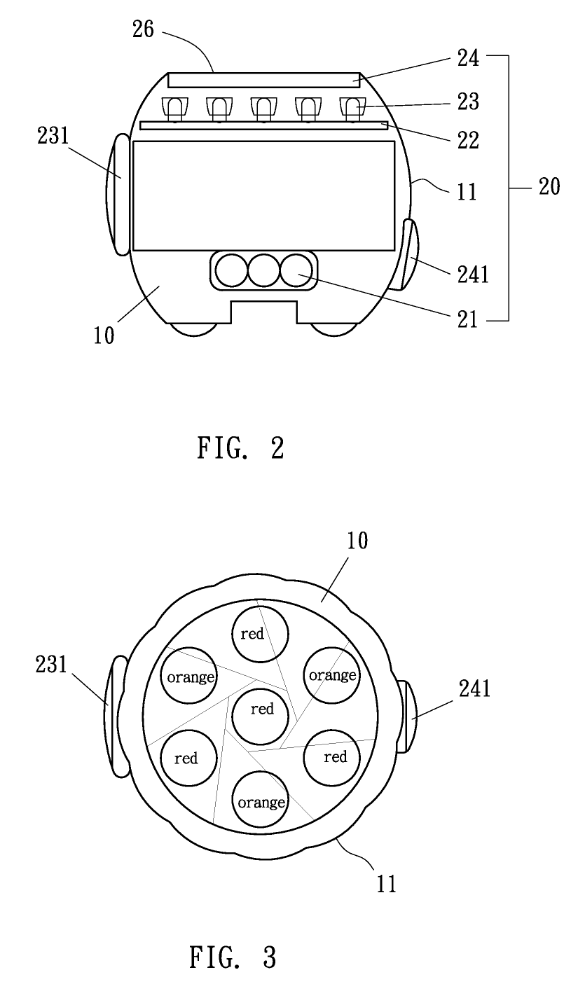

The LEDs 23 are comprised of red LEDs capable of emitting red light having a wavelength in the range of 590 mm to 660 mm, and orange LEDs capable of emitting orange light having a wavelength in the range of 590 mm to 660 mm. As shown in FIG. 2, a red LED is mounted on a center of a circular top of the main body 10 and six alternate red and orange LEDs mounted around the central LED. An illumination area 26 is generated on a top of the aperture 24 when the LEDs 23 are activated. Moreover, a convex (i.e., dome-shaped) optically transmissive lens 12 is formed on the illumination area 26 (see FIG. 1). The provision of the uneven outer surface 11 can facilitate the holding of the illuminating device by the hand in order to easily find a blood vessel (e.g., vein) prior to blood transfusion.

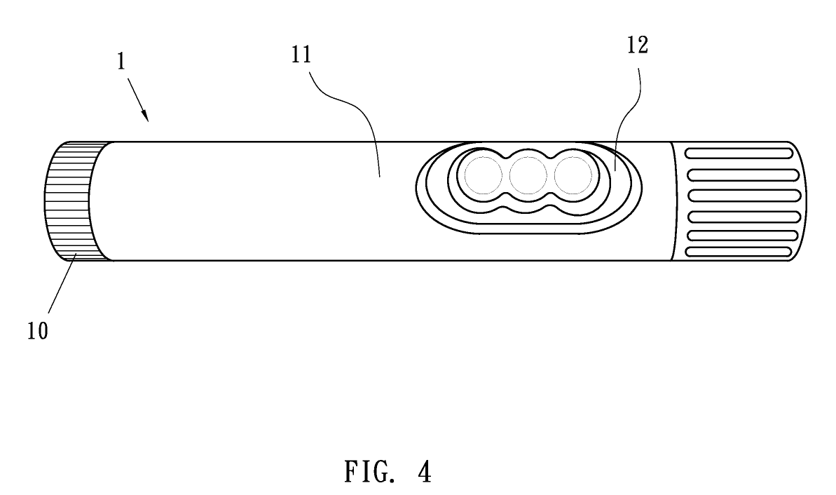

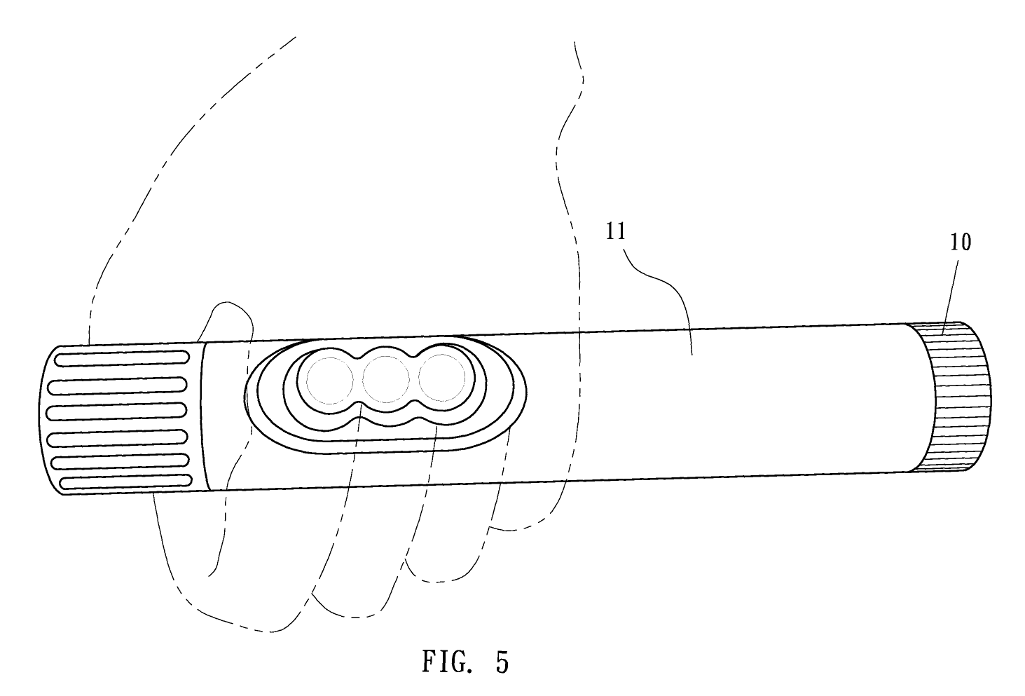

Referring to FIGS. 4 and 5, a hand-held illuminating device 1 for finding blood vessel for blood transfusion in accordance with a second preferred embodiment of the invention is shown. The characteristics of the second preferred embodiment are detailed below. The outer surface 11 of the illuminating device 1 is a smooth cylindrical surface. The convex lens 12 is elliptic when viewing from the top. Also, either end of the main body 10 of the illuminating device 1 is knurled. Both the aperture and its control are eliminated due to narrow space consideration. These characteristics can facilitate holding by the hand.

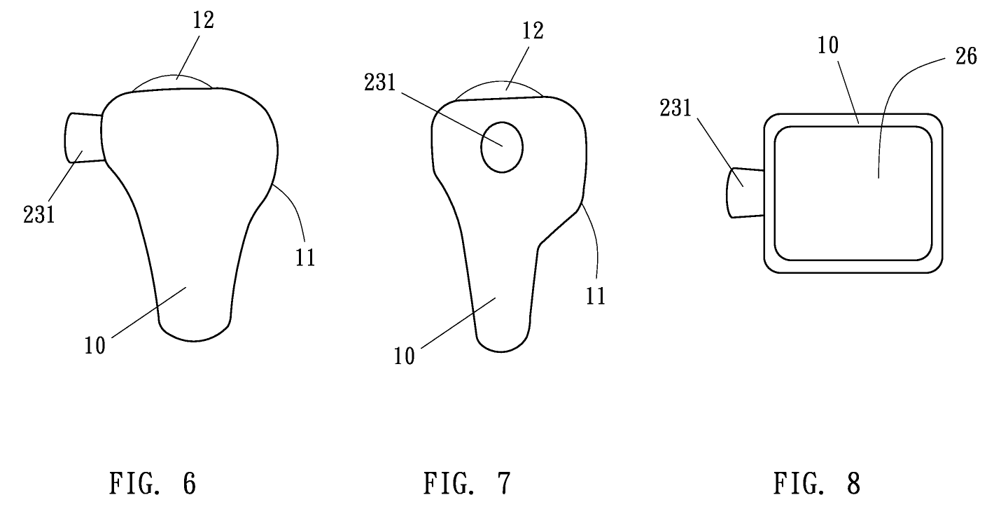

Referring to FIGS. 6 to 8, a hand-held illuminating device for finding blood vessel for blood transfusion in accordance with a third preferred embodiment of the invention is shown. The characteristics of the third preferred embodiment are detailed below. The illuminating device is shaped as a gearshift lever of an automobile. The dim switch 231 is substantially shaped as a short cylinder. Both the aperture and its control are eliminated due to narrow space consideration.

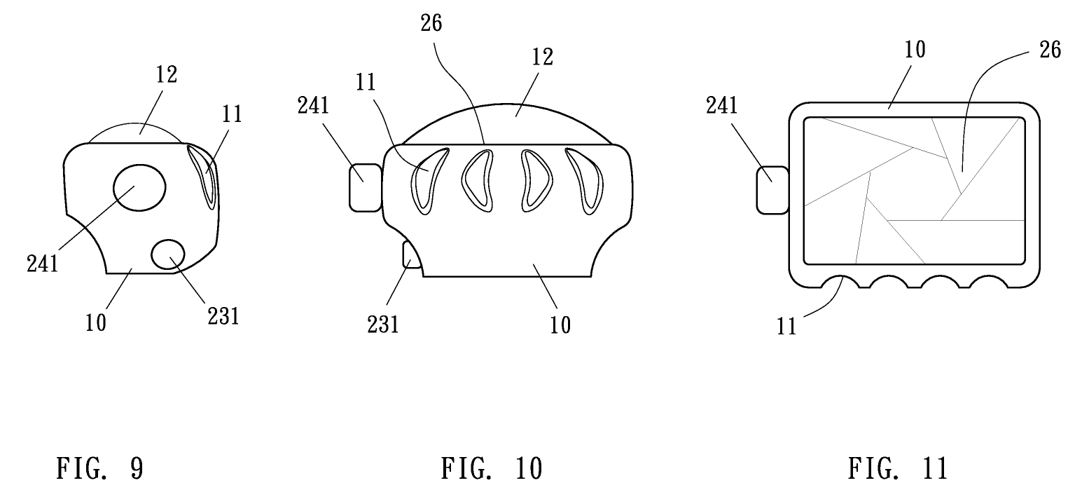

Referring to FIGS. 9 to 11, a hand-held illuminating device for finding blood vessel for blood transfusion in accordance with a fourth preferred embodiment of the invention is shown. The characteristics of the fourth preferred embodiment are detailed below. The illuminating device is shaped as an enlarged head. The control 241 is substantially shaped as a short cylinder. The control 241 is disposed above the dim switch 231. A plurality of (e.g., four) recesses are formed one of four sides of the outer surface 11. A top of the main body 10 is shaped as a rectangle.

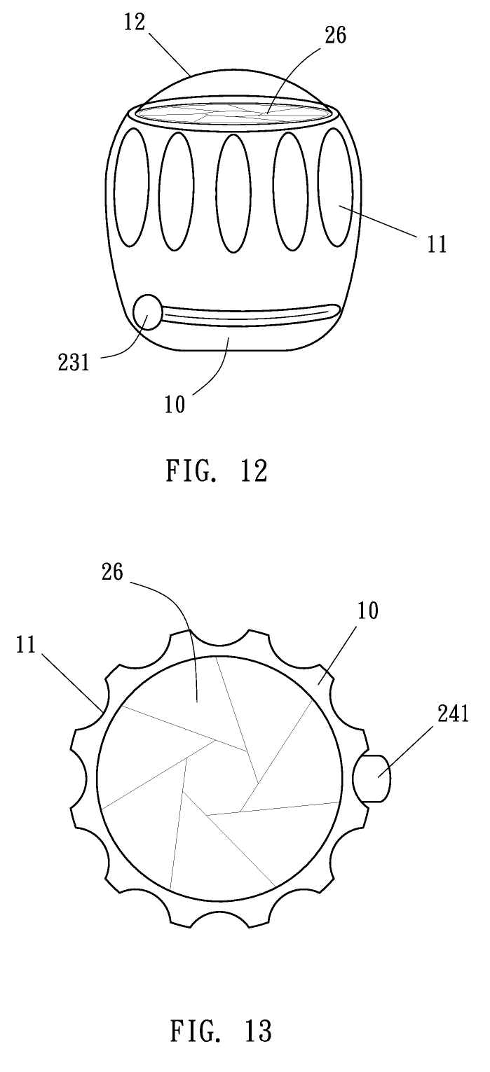

Referring to FIGS. 12 and 13, a hand-held illuminating device for finding blood vessel for blood transfusion in accordance with a fifth preferred embodiment of the invention is shown. The characteristics of the fifth preferred embodiment are detailed below. The outer surface 11 of the main body 10 has a plurality of parallel elliptic indentations therearound.

While the invention herein disclosed has been described by means of specific embodiments, numerous modifications and variations could be made thereto by those skilled in the art without departing from the scope and spirit of the invention set forth in the claims.

What is claimed is:

1. A hand-held illuminating device for finding a blood vessel comprising:
a main body adapted to be stably held by the hand; and
a battery powered illumination assembly in the main body and comprising a circuit board, a plurality of LEDs (light-emitting diodes) mounted on the circuit board and electrically connected thereto, a dim switch on an outer surface of the main body for activating the LEDs or not, and a lens on one end of the main body in proximity to the LEDs,
wherein the LEDs comprises a plurality of first LEDs capable of emitting a first color light having a wavelength in the range of 590 mm to 660 mm, and a plurality of second LEDs capable of emitting a second color light having a wavelength in the range of 590 mm to 660 mm.

2. The hand-held illuminating device of claim 1, further comprising an aperture between the LEDs and the lens, and a control on the outer surface of the main body for adjusting a diameter of the aperture.

3. The hand-held illuminating device of claim 1, wherein the first LEDs are red LEDs and the first color is red.

4. The hand-held illuminating device of claim 1, wherein the second LEDs are orange LEDs and the second color is orange.
